(19) United States
(12) Patent Application Publication
(10) Pub. No.: US 2024/0356073 A1
(43) Pub. Date: Oct. 24, 2024

(54) ELECTROLYTE FOR RECHARGEABLE LITHIUM BATTERY AND RECHARGEABLE LITHIUM BATTERY INCLUDING THE SAME

(71) Applicant: SAMSUNG SDI CO., LTD., Yongin-si (KR)

(72) Inventors: Hanmam JEONG, Yongin-si (KR); Tae Jin LEE, Yongin-si (KR); Si-Young CHA, Yongin-si (KR); Inah KANG, Yongin-si (KR)

(21) Appl. No.: 18/497,693

(22) Filed: Oct. 30, 2023

(30) Foreign Application Priority Data
Apr. 21, 2023 (KR) 10-2023-0052815
Oct. 11, 2023 (KR) 10-2023-0135278

(51) Int. Cl. H01M 10/0567; C07C 69/007; C07D 327/04; C07D 497/10; H01M 10/052

(52) U.S. Cl. CPC H01M 10/0567; C07C 69/007; C07D 327/04; C07D 497/10; H01M 10/052; H01M 2300/0025

(57) ABSTRACT

This disclosure relates to an electrolyte for a rechargeable lithium battery and a rechargeable lithium battery including the same. Some embodiments provide an electrolyte additive for a rechargeable lithium battery including a non-aqueous organic solvent; a lithium salt; a first additive including at least one selected from an additive represented by Chemical Formula 1 and an additive represented by Chemical Formula 2; and a second additive represented by Chemical Formula 3:

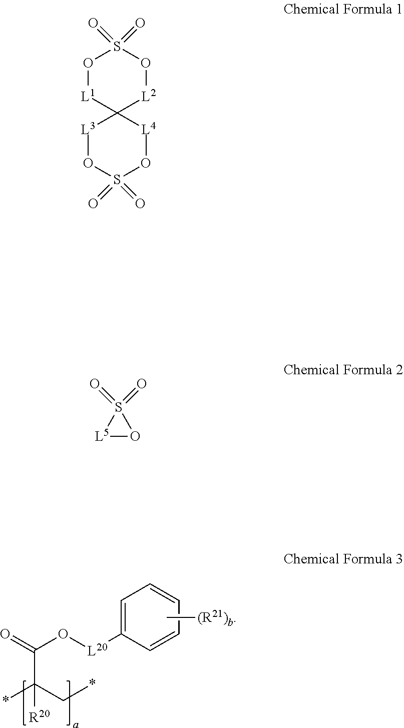

### CROSS-REFERENCE TO RELATED APPLICATION

The present application claims priority to and the benefit of Korean Patent Application No. 10-2023-0052815 filed on Apr. 21, 2023, and Korean Patent Application No. 10-2023-0135278 filed on Oct. 11, 2023, in the Korean Intellectual Property Office, the entire contents of which are herein incorporated by reference.

### BACKGROUND

### 1. Field

Embodiments of this disclosure relate to an electrolyte for a rechargeable lithium battery and a rechargeable lithium battery including the same.

### 2. Description of Related Art

Rechargeable lithium batteries may be recharged and has three or more times as high energy density per unit weight as a lead storage battery, nickel-cadmium battery, nickel hydrogen battery, nickel zinc battery and the like. They may be also charged at a high rate and thus, are commercially manufactured for a laptop, a cell phone, an electric tool, an electric bike, and the like, and research on improvement of additional energy density of such rechargeable lithium batteries have been actively made.

Such rechargeable lithium batteries may be manufactured by injecting an electrolyte into an electrode assembly, which includes a positive electrode including a positive electrode active material capable of intercalating/deintercalating lithium ions and a negative electrode including a negative electrode active material capable of intercalating/deintercalating lithium ions.

Recently, one development direction of rechargeable lithium batteries is to improve high-temperature characteristics. In general, rechargeable lithium batteries may have problems of an increase in resistance, ignition, and/or explosion at a high temperature. For example, in a module and/or a pack manufactured by assembling several rechargeable lithium battery cells, if one rechargeable lithium battery cell starts to ignite and/or explode, heat is propagated in sequence to adjacent cells, resulting in igniting and/or exploding the entire module and/or pack.

### SUMMARY

An electrolyte for a rechargeable lithium battery according to some embodiments is intended to suppress or reduce ignition and/or explosion of a corresponding cell at a high temperature, and to prevent or reduce an increase in temperature of a corresponding cell even if ignition and/or explosion starts in an adjacent cell.

Some embodiments provide an electrolyte additive for a rechargeable lithium battery including a non-aqueous organic solvent; a lithium salt; a first additive including at least one selected from an additive represented by Chemical Formula 1 and an additive represented by Chemical Formula 2; and a second additive represented by Chemical Formula 3:

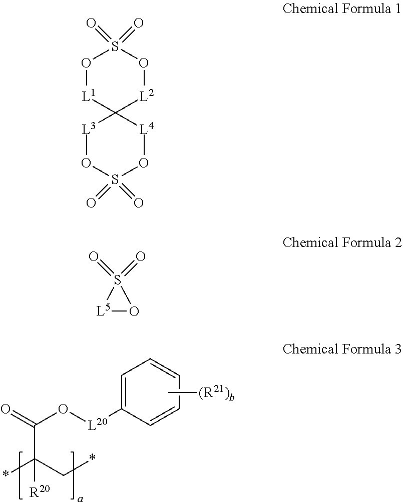

### BRIEF DESCRIPTION OF THE DRAWINGS

The accompanying drawings, together with the specification, illustrate embodiments of the subject matter of the present disclosure, and, together with the description, serve to explain principles of embodiments of the subject matter of the present disclosure.

FIG. 1 is a schematic view showing a rechargeable lithium battery according to some embodiments.

FIGS. 2-3 show heat exposure evaluation results of the rechargeable lithium battery cells of Comparative Example 1 and Example 1 when exposed to heat of 140° C., respectively.

FIGS. 4-7 show heat exposure evaluation results of the rechargeable lithium battery cells of Comparative Examples 1, 5, and 10 and Example 19 when exposed to heat of 142° C., respectively.

### DETAILED DESCRIPTION

Hereinafter, a rechargeable lithium battery according to some embodiments will be described in more detail with reference to the accompanying drawings. However, these embodiments are examples, the present disclosure is not limited thereto and the scope of the present disclosure is defined by the scope of claims, and equivalents thereof.

As used herein, if a specific definition is not otherwise provided, “substituted” refers to replacement of at least one hydrogen of a compound by a halogen atom (F, Cl, Br, or I), a hydroxy group, a C1 to C20 alkoxy group, a nitro group, a cyano group, an amine group, an imino group, an azido group, an amidino group, a hydrazino group, a hydrazono group, a carbonyl group, a carbamyl group, a thiol group, an ester group, an ether group, a carboxyl group or a salt thereof, a sulfonic acid group or a salt thereof, a phosphoric acid or a salt thereof, a C1 to C20 alkyl group, a C2 to C20 alkenyl group, a C2 to C20 alkynyl group, a C6 to C30 aryl group, a C3 to C20 cycloalkyl group, a C3 to C20 cycloalkenyl group, a C3 to C20 cycloalkynyl group, a C2 to C20 heterocycloalkyl group, a C2 to C20 heterocycloalkenyl group, a C2 to C20 heterocycloalkynyl group, or a combination thereof.

As used herein, if a specific definition is not otherwise provided, “heterocycloalkyl group”, “heterocycloalkenyl group”, “heterocycloalkynyl group,” and “heterocycloalkylene group” refer to presence of at least one N, O, S, or P in a cyclic compound of cycloalkyl, cycloalkenyl, cycloalkynyl, and cycloalkylene.

In the chemical formula of the present specification, unless a specific definition is otherwise provided, hydrogen is bonded at the position if a chemical bond is not drawn where supposed to be given.

As used herein, if a definition is not otherwise provided, “*” refers to a linking part between the same or different atoms, or chemical formulas.

As used herein, if a specific definition is not otherwise provided, “weight average molecular weight” is measured by a gel permeation chromatography (GPC).

### Electrolyte

Some embodiments provide an electrolyte for a rechargeable lithium battery including a non-aqueous organic solvent; a lithium salt; a first additive including at least one selected from an additive represented by Chemical Formula 1 and an additive represented by Chemical Formula 2; and a second additive represented by Chemical Formula 3:

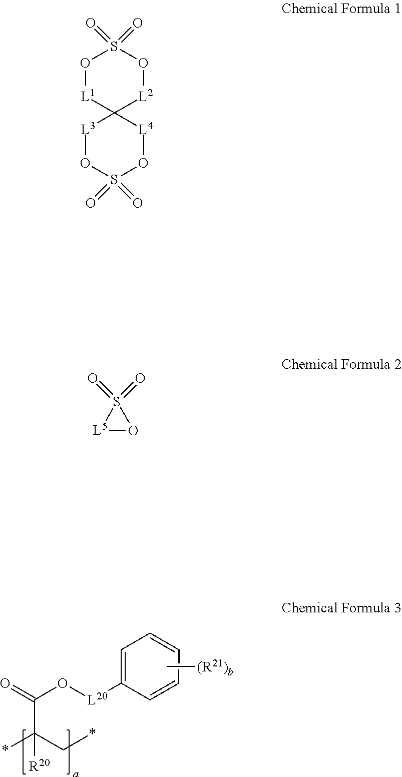

The first additive is a cyclic monomolecular compound having a sulfate group, which may be reduced and decomposed on the negative electrode at a high temperature of about 100° C. or higher to form a film. In some embodiments, the reduced material may migrate to the positive electrode to form an S-based solid electrolyte interface (SEI) of RSO₃Li/RSO₂Li, which has an effect of suppressing or reducing an increase in resistance by suppressing or reducing the formation of P-based reaction products and an increase of LiF.

The second additive is a polymer, and π-π stacking of benzene rings is formed inside at least one polymer at a high temperature exceeding about 140° C. As a result, an internal binding force is generated inside at least one polymer, and a gelation phenomenon occurs as the different polymers having the internal binding force are agglomerated.

Overall, the combination of the two additives has an effect of shutting down the rechargeable lithium battery cell by rapidly increasing viscosity of the electrolyte at a high temperature exceeding 140° C. and concurrently (e.g., simultaneously) reducing the ionic conductivity.

Therefore, the rechargeable lithium battery to which the electrolyte for a rechargeable lithium battery according to some embodiments is applied can suppress or reduce ignition and/or explosion of the corresponding cell at a high temperature by combining the two additives, and even if ignition and/or explosion starts in an adjacent cell, the temperature increase of the corresponding cell can be prevented or reduced.

Hereinafter, an electrolyte for a rechargeable lithium battery according to some embodiments will be described in more detail.

### First Additive

The descriptions of Chemical Formula 1 and Chemical Formula 2 representing the first additive are as follows:

In Chemical Formula 1, L¹ to L⁴ are each independently a single bond or a substituted or unsubstituted C1 to C10 alkylene group.

In some embodiments, L¹ to L⁴ may all be substituted or unsubstituted C1 to C10 alkylene groups.

In some embodiments, L¹ to L⁴ may all be methylene groups.

In Chemical Formula 2, L⁵ is a single bond or a substituted or unsubstituted C1 to C10 alkylene group.

In some embodiments, L⁵ may be a substituted or unsubstituted C1 to C10 alkylene group.

In some embodiments, L⁵ may be a methylene group.

Representative, non-limiting examples of the first additive are as follows:

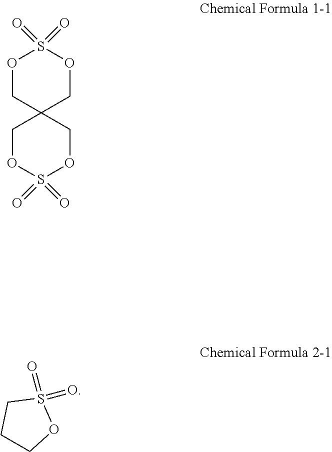 [SMILES: O=S1(=O)OCC2(CO1)COS(=O)(=O)OC2 ; O=S1(=O)CCCO1]

### Second Additive

The descriptions of Chemical Formula 3 representing the second additive are as follows:
R²⁰ and R²¹ are each independently a hydrogen atom, a halogen atom, a substituted or unsubstituted C1 to C10 alkyl group, a vinyl group, a substituted or unsubstituted C6 to C10 aryl group, or a substituted or unsubstituted C2 to C10 heteroaryl group including a N, O, or P heteroatom; L²⁰ is a single bond or a substituted or unsubstituted C1 to C10 alkylene group; a is an integer of 1 to 100; and b is an integer of 0 to 5.

In some embodiments, R²⁰ and R²¹ may each independently be a hydrogen atom; or a substituted or unsubstituted C1 to C10 alkyl group.

In some embodiments, both R²⁰ and R²¹ may be a methyl group.

In some embodiments, b may be 0.

In some embodiments, L²⁰ may be a substituted or unsubstituted C1 to C10 alkylene group.

In some embodiments, L²⁰ may be a methylene group.

A representative, non-limiting example of the second additive is as follows:

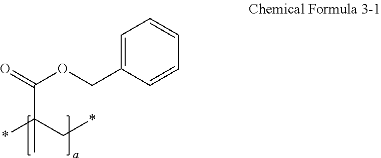

wherein, in Chemical Formula 3-1, a is an integer of 1 to 100.

In some embodiments, a weight average molecular weight of the second additive may be about 5,000 to about 1,000,000 g/mol.

In some embodiments, the weight average molecular weight of the second additive may be about 10,000 to about 500,000 g/mol.

### Weight Ratio of First Additive and Second Additive

In some embodiments, the weight ratio of the first additive to the second additive may be about 1:2 to about 1:20. Within these ranges, there is a synergistic effect due to the combination of the two additives.

In some embodiments, the weight ratio of the first additive and the second additive may be about 1:2 to about 1:10, or about 1:3 to about 1:6.

### Content of Each Additive

In some embodiments, the first additive may be included in an amount of about 0.10 to about 2.00 wt % based on a total amount of the electrolyte. Within this range, the effect of the first additive may be increased.

In some embodiments, the first additive may be included in an amount of about 0.25 to about 1.00 wt % based on a total amount of the electrolyte.

In some embodiments, the second additive may be included in an amount of about 1.00 to about 15.00 wt % based on a total amount of the electrolyte. Within this range, the effect of the second additive may be increased.

In some embodiments, the second additive may be included in an amount of about 3.00 to about 10.00 wt % based on a total amount of the electrolyte.

In some embodiments, the weight ratio of the first additive to the second additive may be about 1:2 to about 1:20. Within this range, there is a synergistic effect due to the combination of the two additives.

In some embodiments, the weight ratio of the first additive and the second additive may be about 1:2 to about 1:10, or about 1:3 to about 1:6.

### Non-Aqueous Organic Solvent

The non-aqueous organic solvent serves as a medium for transmitting ions taking part in the electrochemical reaction of a battery.

The non-aqueous organic solvent may include a carbonate-based, ester-based, ether-based, ketone-based, alcohol-based, and/or aprotic solvent.

The carbonate-based solvent may include ethylmethyl carbonate (EMC), dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), and/or the like. The ester-based solvent may include methyl acetate, ethyl acetate, n-propyl acetate, t-butyl acetate, methylpropionate, ethylpropionate, propylpropionate, decanolide, mevalonolactone, caprolactone, and/or the like. The ether-based solvent may include dibutyl ether, tetraglyme, diglyme, dimethoxyethane, 2-methyltetrahydrofuran, tetrahydrofuran, and/or the like. The ketone-based solvent may include cyclohexanone and/or the like. The alcohol-based solvent may include ethyl alcohol, isopropyl alcohol, and/or the like, and examples of the aprotic solvent include nitriles such as R—CN (wherein R is a C2 to C20 linear, branched, or cyclic hydrocarbon group, a double bond, an aromatic ring, or an ether bond), amides such as dimethylformamide, dioxolanes such as 1,3-dioxolane, sulfolanes, and/or the like.

The non-aqueous organic solvent may be used alone or in combination with one or more of them, and if used in combination with one or more, a mixing ratio may be suitably appropriately adjusted according to suitable or desired battery performance, which is well understood by those skilled in the art.

The carbonate-based solvent is prepared by mixing together a cyclic carbonate and a chain carbonate. The cyclic carbonate and chain carbonate are mixed together in a volume ratio of about 5:95 to about 50:50. If the mixture is used as an electrolyte, it may have enhanced performance.

In some embodiments, ethylene carbonate (EC) may be used as the cyclic carbonate, and ethylmethyl carbonate (EMC) and dimethyl carbonate (DMC) may be used as the chain carbonate.

In some embodiments, the non-aqueous organic solvent may include a carbonate-based solvent in which ethylene carbonate (EC), ethylmethyl carbonate (EMC), and dimethyl carbonate (DMC) are mixed together. For example, the carbonate-based solvent in which ethylene carbonate (EC), ethylmethyl carbonate (EMC), and dimethyl carbonate (DMC) are mixed together is mixed together in a volume ratio of EC:EMC:DMC=about 1:0.5:5 to about 5:3:10, which may improve performance of the electrolyte.

The non-aqueous organic solvent may further include an aromatic hydrocarbon-based solvent as well as the carbonate-based solvent. The carbonate-based solvent and aromatic hydrocarbon-based solvent may be mixed together in a volume ratio of about 1:1 to about 30:1.

The aromatic hydrocarbon-based solvent may be an aromatic hydrocarbon-based compound represented by Chemical Formula I.

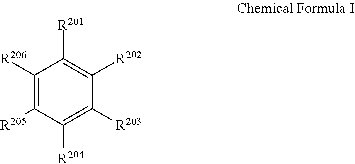

In Chemical Formula I, R²⁰¹ to R²⁰⁶ are the same or different and are selected from hydrogen, a halogen, a C1 to C10 alkyl group, a haloalkyl group, and a combination thereof.

Examples of the aromatic hydrocarbon-based solvent may be selected from benzene, fluorobenzene, 1,2-difluorobenzene, 1,3-difluorobenzene, 1,4-difluorobenzene, 1,2,3-trifluorobenzene, 1,2,4-trifluorobenzene, chlorobenzene, 1,2-dichlorobenzene, 1,3-dichlorobenzene, 1,4-dichlorobenzene, 1,2,3-trichlorobenzene, 1,2,4-trichlorobenzene, iodobenzene, 1,2-diiodobenzene, 1,3-diiodobenzene, 1,4-diiodobenzene, 1,2,3-triiodobenzene, 1,2,4-triiodobenzene, toluene, fluorotoluene, 2,3-difluorotoluene, 2,4-difluorotoluene, 2,5-difluorotoluene, 2,3,4-trifluorotoluene, 2,3,5-trifluorotoluene, chlorotoluene, 2,3-dichlorotoluene, 2,4-dichlorotoluene, 2,5-dichlorotoluene, 2,3,4-trichlorotoluene, 2,3,5-trichlorotoluene, iodotoluene, 2,3-diiodotoluene, 2,4-diiodotoluene, 2,5-diiodotoluene, 2,3,4-triiodotoluene, 2,3,5-triiodotoluene, xylene, and a combination thereof.

The electrolyte may further include vinylene carbonate, vinyl ethylene carbonate, and/or an ethylene-based carbonate-based compound represented by Chemical Formula II to improve cycle-life of a battery as a cycle life-enhancing additive.

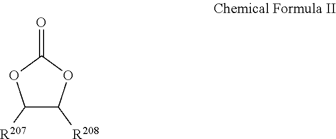

In Chemical Formula II, R²⁰⁷ and R²⁰⁸ are the same or different and are selected from hydrogen, a halogen, a cyano group (CN), a nitro group (NO₂), and a C1 to C5 fluoroalkyl group, provided that at least one selected from R²⁰⁷ and R²⁰⁸ is a halogen, a cyano group (CN), a nitro group (NO₂), or a C1 to C5 fluoroalkyl group, and R²⁰⁷ and R²⁰⁸ are not simultaneously hydrogen.

Examples of the ethylene carbonate-based compound include difluoro ethylenecarbonate, chloroethylene carbonate, dichloroethylene carbonate, bromoethylene carbonate, dibromoethylene carbonate, nitroethylene carbonate, cyanoethylene carbonate or fluoroethylene carbonate. The amount of the additive for improving cycle-life may be used within a suitable or appropriate range.

### Lithium Salt

The lithium salt is dissolved in a non-aqueous organic solvent, supplies a battery with lithium ions, basically operates the rechargeable lithium battery, and improves transportation of the lithium ions between positive and negative electrodes. Examples of the lithium salt include one or more selected from LiPF₆, LiBF₄, LiDFOP, LIDFOB, LiPO₂F₂, LiSbF₆, LiAsF₆, LIN (SO₂C₂F₅)₂, Li(CF₃SO₂)₂N, LiN(SO₃C₂F₅)₂, Li(FSO₂)₂N (lithium bis(fluorosulfonyl)imide: LiFSI), LiC₄F₉SO₃, LiClO₄, LiAlO₂, LiAlCl₄, LIN (CₓF₂ₓ₊₁SO₂) (C_{y}F_{2y+1}SO₂), (where x and y are natural numbers, for example an integer of 1 to 20), LiCl, LiI and LiB(C₂O₄)₂ (lithium bis(oxalato)borate; LiBOB). The lithium salt may be used in a concentration in a range from about 0.1 M to about 2.0 M. If the lithium salt is included at the above concentration range, an electrolyte may have excellent performance and lithium ion mobility due to suitable or optimal electrolyte conductivity and viscosity.

### Rechargeable Lithium Battery

Some embodiments provide a rechargeable lithium battery including a positive electrode; a negative electrode; and the aforementioned additive according to some embodiments, or the aforementioned electrolyte according to some embodiments.

As the rechargeable lithium battery of some embodiments includes the aforementioned electrolyte according to some embodiments, it is possible to suppress or reduce ignition and/or explosion of the corresponding cell at a high temperature, and even if ignition and/or explosion starts in an adjacent cell, the temperature increase of the corresponding cell can be prevented or reduced.

Hereinafter, descriptions overlapping with those described above will not be repeated, and the rechargeable lithium battery will be described in more detail.

### Positive Electrode

The positive electrode includes a positive electrode current collector and a positive electrode active material layer on the positive electrode current collector, and the positive electrode active material layer includes a positive electrode active material.

The positive electrode active material may include lithiated intercalation compounds that reversibly intercalate and deintercalate lithium ions.

For example, at least one composite oxide of lithium and a metal of cobalt, manganese, nickel, or a combination thereof may be used.

The composite oxide may have a coating layer on the surface thereof and may be used, or a mixture of the composite oxide and the composite oxide having a coating layer may be used. The coating layer may include a coating element compound selected from oxide of the coating element, hydroxide of the coating element, oxyhydroxide of the coating element, oxycarbonate of the coating element, or hydroxycarbonate of the coating element. The compound for the coating layer may be either amorphous or crystalline. The coating element included in the coating layer may be Mg, Al, Co, K, Na, Ca, Si, Ti, V, Sn, Ge, Ga, B, As, Zr, or a mixture thereof. The coating process may include any suitable processes generally used in the art as long as it does not cause any side effects (e.g., does not cause substantially any undesirable side effects) on the properties of the positive electrode active material (e.g., spray coating, dipping), and thus, a further detailed description thereof is not necessary here.

In some embodiments, the positive electrode active material may include a lithium nickel-based composite oxide represented by Chemical Formula A1:

Liₐ₁Niₓ₁M¹_{y1}M²_{z1}O_{2-b1}X_{b1} Chemical Formula A1
wherein, in Chemical Formula A1, 0.9≤a1≤1.2, 0.7≤x1≤1, 0≤y1≤0.2, 0≤z1≤0.2, 0.9≤x1+y1+z1≤1.1, and 0≤b1≤0.1; M1 and M2 are each independently one or more elements selected from Al, B, Ba, Ca, Ce, Co, Cr, Cu, Fe, Mg, Mn, Mo, Nb, Si, Sr, Ti, V, W, and Zr; and X is one or more elements selected from F, P, and S.

In Chemical Formula A1, 0.75≤x1≤1, 0≤y1≤0.18, and 0≤z1≤0.18; 0.85≤x1≤1, 0≤y1≤0.15, and 0≤z1≤0.15; or 0.9≤x1≤1, 0≤y1≤0.1, and 0≤z1≤0.1.

For example, the positive electrode active material may include a lithium nickel-based composite oxide represented by Chemical Formula A2. The compound represented by Chemical Formula A2 may be referred to as a lithium nickel cobalt-based composite oxide:

Liₐ₂Niₓ₂CO_{y2}M³_{z2}O_{2-b2}X_{b2} Chemical Formula A2
wherein, in Chemical Formula A2, 0.9≤a2≤1.8, 0.7≤x2<1, 0<y2≤0.2, 0≤z2≤0.2, 0.9≤x2+y2+z2≤1.1, and 0≤b2≤0.1, M3 is one or more elements selected from Al, B, Ba, Ca, Ce, Cr, Fe, Mg, Mn, Mo, Nb, Si, Sr, Ti, V, W, and Zr, and X is one or more elements selected from F, P, and S.

In Chemical Formula A2, 0.75≤x2≤0.99, 0≤y2≤0.15, and 0≤z2≤0.15; 0.85≤x2≤0.99, 0.01≤y2≤0.15, and 0.01≤z2≤0.15; or 0.9≤x2≤0.99, 0.01≤y2≤0.1, and 0.01≤z2≤0.1.

For example, the positive electrode active material may include a lithium nickel-based composite oxide represented by Chemical Formula A3. The compound represented by Chemical Formula A3 may be referred to as lithium nickel-cobalt-aluminum oxide or lithium nickel-cobalt-manganese oxide.

Liₐ₃Niₓ₃CO_{y3}M⁴_{z3}M⁵_{w3}O_{2-b3}X_{b3} Chemical Formula A3

In Chemical Formula A3, 0.9≤a3≤1.8, 0.7≤x3≤0.98, 0.01≤y3≤0.19, 0.01≤z3≤0.19, 0≤w3≤0.19, 0.9≤x3+y3+z3+w3≤1.1, and 0≤b3≤0.1, M⁴ is one or more elements selected from A1 and Mn, M5 is one or more elements selected from B, Ba, Ca, Ce, Cr, Fe, Mg, Mo, Nb, Si, Sr, Ti, V, W, and Zr, and X is one or more elements selected from F, P, and S.

In Chemical Formula A3, 0.75≤x3≤0.98, 0≤y3≤0.16, and 0≤z3≤0.16; 0.85≤x3≤0.98, 0.01≤y3≤0.14, 0.01≤z3≤0.14, and 0≤w3≤0.14; or 0.9≤x3≤0.98, 0.01≤y3≤0.09, 0.01≤z3≤0.09, and 0≤w3≤0.09.

For example, the positive electrode active material may include a lithium nickel-based composite oxide represented by Chemical Formula A4. The compound represented by Chemical Formula A4 may be referred to as a cobalt-free lithium nickel-manganese oxide.

Liₐ₄Niₓ₄Mn_{y4}M⁶_{z4}O_{2-b4}X_{b4} Chemical Formula A4

In Chemical Formula A4, 0.9≤a2≤1.8, 0.7≤x4<1, 0<y4≤0.2, 0≤z4≤0.2, 0.9≤x4+y4+z4≤1.1, and 0≤b4≤0.1 M6 is one or more elements selected from Al, B, Ba, Ca, Ce, Cr, Fe, Mg, Mo, Nb, Si, Sr, Ti, V, W, and Zr, and X is one or more elements selected from F, P, and S.

In a positive electrode according to some embodiments, a content of the positive electrode active material may be about 50 wt % to about 99 wt %, about 60 wt % to about 99 wt %, about 70 wt % to about 99 wt %, about 80 wt % to about 99 wt %, or about 90 wt % to about 99 wt % based on a total weight of the positive electrode active material layer.

In some embodiments of the present disclosure, the positive electrode active material layer may optionally include a conductive material (e.g., electrically conductive material) and a binder. In some embodiments, each content of the conductive material and the binder may be about 1.0 wt % to about 5.0 wt %, based on a total weight of the positive electrode active material layer.

The conductive material is used to impart conductivity (e.g., electrical conductivity) to the negative electrode, and any suitable electrically conductive material may be used as the conductive material unless it causes a chemical change in a battery (e.g., an undesirable chemical change in the battery). Examples of the conductive material may include a carbon-based material such as natural graphite, artificial graphite, carbon black, acetylene black, ketjen black, carbon fiber, and the like; a metal-based material of a metal powder or a metal fiber including copper, nickel, aluminum, silver, and the like; a conductive polymer such as a polyphenylene derivative; or a mixture thereof.

The binder improves binding properties of positive electrode active material particles with one another and with a current collector and examples thereof may be polyvinyl alcohol, carboxylmethyl cellulose, hydroxypropyl cellulose, diacetyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, an acrylated styrene-butadiene rubber, an epoxy resin, nylon, and the like, but are not limited thereto.

A1 may be used as the positive electrode current collector, but is not limited thereto.

### Negative Electrode

The negative electrode includes a negative electrode current collector and a negative electrode active material layer including the negative electrode active material on the negative electrode current collector.

The negative electrode active material may be a material that reversibly intercalates/deintercalates lithium ions, a lithium metal, a lithium metal alloy, a material capable of doping and dedoping lithium, and/or a transition metal oxide.

The material that reversibly intercalates/deintercalates lithium ions includes carbon materials. The carbon material may be any suitable carbon-based negative electrode active material generally used in the art in a rechargeable lithium battery. Examples of the carbon material include crystalline carbon, amorphous carbon, and a combination thereof. The crystalline carbon may be non-shaped, and/or sheet, flake, spherical, and/or fiber shaped natural graphite and/or artificial graphite. The amorphous carbon may be a soft carbon, a hard carbon, a mesophase pitch carbonized product, fired coke, and/or the like.

The lithium metal alloy may include lithium and a metal selected from Na, K, Rb, Cs, Fr, Be, Mg, Ca, Sr, Si, Sb, Pb, In, Zn, Ba, Ra, Ge, A1, and Sn.

The material capable of doping and dedoping lithium may include Si, SiOₓ (0<x<2), a Si-Q alloy (wherein Q is selected from an alkali metal, an alkaline-earth metal, a Group 13 element, a Group 14 element, a Group 15 element, a Group 16 element, a transition metal, a rare earth element, and a combination thereof, and not Si), Sn, SnO₂, a Sn—R alloy (wherein R is an alkali metal, an alkaline-earth metal, a Group 13 element, a Group 14 element, a Group 15 element, a Group 16 element, a transition element, a rare earth element, or a combination thereof, and not Sn), and/or the like. At least one of them may be mixed together with SiO₂.

The elements Q and R may be selected from Mg, Ca, Sr, Ba, Ra, Sc, Y, Ti, Zr, Hf, Rf, V, Nb, Ta, Db, Cr, Mo, W, Sg, Tc, Re, Bh, Fe, Pb, Ru, Os, Hs, Rh, Ir, Pd, Pt, Cu, Ag, Au, Zn, Cd, B, Al, Ga, Sn, In, TI, Ge, P, As, Sb, Bi, S, Se, Te, Po, and a combination thereof.

The transition metal oxide may be a vanadium oxide, a lithium vanadium oxide, and/or the like.

In some embodiments, the negative electrode active material may include at least one selected from graphite and a Si composite.

The Si composite may include a core including Si particles and amorphous carbon, for example, the Si particles may include at least one selected from Si composite, SiOₖ (0<k≤2), and an Si alloy.

For example, the Si—C composite may include a core including Si particles and amorphous carbon.

The central portion of the core may include pores, and the radius of the central portion may correspond to about 30% to about 50% of the radius of the Si—C composite.

The Si particles may have an average particle diameter of about 10 nm to about 200 nm.

As used herein, the average particle diameter may be a particle size (D50) at a volume ratio of 50% in a cumulative size-distribution curve.

If the average particle diameter of the Si particle is within the above range, volume expansion occurring during charging and discharging may be suppressed or reduced, and a disconnection of a conductive path due to particle crushing during charging and discharging may be prevented or reduced.

The Si particle may be included in an amount of about 1 wt % to about 60 wt %, for example, about 3 wt % to about 60 wt %, based on a total weight of the Si—C composite.

The central portion of the negative electrode active material may not include amorphous carbon, but the amorphous carbon may be present only on the surface portion of the negative electrode active material.

Herein, the surface portion indicates a region from the central portion of the negative electrode active material to the outermost surface of the negative electrode active material.

In some embodiments, the Si particles are substantially uniformly included over the negative electrode active material, for example, present at a substantially uniform concentration in the central portion and the surface portion thereof.

The amorphous carbon may be soft carbon, hard carbon, mesophase pitch carbonized product, calcined coke, or a combination thereof.

The negative electrode active material may further include crystalline carbon.

If the negative electrode active material includes a Si—C composite and crystalline carbon together, the Si—C composite and crystalline carbon may be included in the form of a mixture, and in some embodiments, the Si—C composite and crystalline carbon may be included in a weight ratio of about 1:99 to about 50:50. In some embodiments, the Si—C composite and crystalline carbon may be included in a weight ratio of about 3:97 to about 20:80 or about 5:95 to about 20:80.

The crystalline carbon may be for example graphite, and, for example, natural graphite, artificial graphite, or a mixture thereof.

The crystalline carbon may have an average particle diameter of about 5 μm to about 30 μm.

The amorphous carbon precursor may include a coal-based pitch, mesophase pitch, petroleum-based pitch, coal-based oil, petroleum-based heavy oil, and/or a polymer resin such as a phenol resin, a furan resin, and/or a polyimide resin.

In the negative electrode active material layer, the negative electrode active material may be included in an amount of about 95 wt % to about 99 wt % based on a total weight of the negative electrode active material layer.

In some embodiments, the negative electrode active material layer may include a binder, and, optionally, a conductive material (e.g., an electrically conductive material). In the negative electrode active material layer, the amount of the binder may be about 1 wt % to about 5 wt % based on a total weight of the negative electrode active material layer. If it further includes the conductive material, it may include about 90 wt % to about 98 wt % of the negative electrode active material, about 1 wt % to about 5 wt % of the binder, and about 1 wt % to about 5 wt % of the conductive material.

The binder improves binding properties of negative electrode active material particles with one another and with a current collector. The binder may be a non-water-soluble binder, a water-soluble binder, or a combination thereof.

The non-water-soluble binder may be polyvinylchloride, carboxylated polyvinylchloride, polyvinylfluoride, polyurethane, polytetrafluoroethylene, polyvinylidene fluoride, polyethylene, polypropylene, polyamideimide, polyimide, or a combination thereof.

The water-soluble binder may be a rubber-based binder and/or a polymer resin binder. The rubber-based binder may be selected from a styrene-butadiene rubber, an acrylated styrene-butadiene rubber (SBR), an acrylonitrile-butadiene rubber, an acrylic rubber, a butyl rubber, a fluorine rubber, and a combination thereof. The polymer resin binder may be selected from polytetrafluoroethylene, ethylenepropylene copolymer, polyethyleneoxide, polyvinylpyrrolidone, polyepichlorohydrine, polyphosphazene, polyacrylonitrile, polystyrene, an ethylene propylenediene copolymer, polyvinylpyridine, chlorosulfonatedpolyethylene, latex, a polyester resin, an acrylic resin, a phenolic resin, an epoxy resin, polyvinylalcohol, and a combination thereof.

If the water-soluble binder is used as the negative electrode binder, a cellulose-based compound may be further used to provide viscosity as a thickener. The cellulose-based compound includes one or more of carboxylmethyl cellulose, hydroxypropylmethyl cellulose, methyl cellulose, and/or alkali metal salts thereof. The alkali metal may be Na, K, and/or Li. Such a thickener may be included in an amount of about 0.1 to about 3 wt % based on 100 wt % of the negative electrode active material.

The conductive material is included to provide electrode conductivity and any suitable electrically conductive material may be used as a conductive material unless it causes a chemical change (e.g., an undesirable chemical change in the rechargeable lithium battery). Examples thereof may be a carbon-based material such as natural graphite, artificial graphite, carbon black, acetylene black, ketjen black, carbon fiber and the like; a metal-based material such as a metal powder and/or a metal fiber of copper, nickel, aluminum, silver, and the like; a conductive polymer such as a polyphenylene derivative and the like, or a mixture thereof.

The negative electrode current collector may be selected from a copper foil, a nickel foil, a stainless steel foil, a titanium foil, a nickel foam, a copper foam, a polymer substrate coated with a conductive metal, and a combination thereof.

### Separator

The rechargeable lithium battery may further include a separator between the negative electrode and the positive electrode, depending on a type (or kind) of the rechargeable lithium battery. These separators are porous substrates; or it may be a composite porous substrate.

The porous substrate may be a substrate including pores, and lithium ions may move through the pores. The porous substrate may be for example may include polyethylene, polypropylene, polyvinylidene fluoride, and multi-layers thereof such as a polyethylene/polypropylene double-layered separator, a polyethylene/polypropylene/polyethylene triple-layered separator, and/or a polypropylene/polyethylene/polypropylene triple-layered separator.

The composite porous substrate may have a form including a porous substrate and a functional layer on the porous substrate. The functional layer may be, for example, at least one selected from a heat-resistant layer and an adhesive layer from the viewpoint of enabling additional function. For example, the heat-resistant layer may include a heat-resistant resin and, optionally, a filler.

In some embodiments, the adhesive layer may include an adhesive resin and, optionally, a filler.

The filler may be an organic filler and/or an inorganic filler.

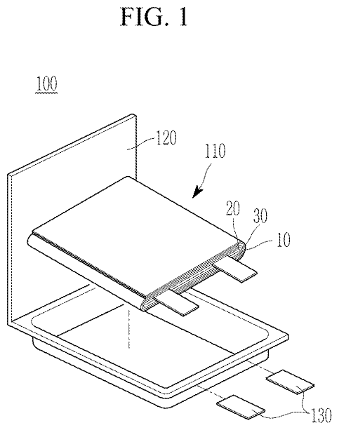

Referring to FIG. 1, a rechargeable lithium battery 100 according to some embodiments includes an electrode assembly 110 wound with a separator 30 between the positive electrode 10 and the negative electrode 20, a case 120 housing the electrode assembly 110, and an electrode tab 130 serving as an electrical passage for inducing the current formed in the electrode assembly 110 to the outside. Both sides of the case 120 are overlapped and sealed. In some embodiments, the electrolyte is injected into the case 120 containing the electrode assembly 110, and the positive electrode 10, the negative electrode 20, and the separator 30 may be impregnated with the electrolyte.

Hereinafter, examples of the present disclosure and comparative examples are described. These examples, however, are not in any sense to be interpreted as limiting the scope of the present disclosure.

### EXAMPLE 1

### (1) Preparation of Electrolytes

As a non-aqueous organic solvent, a carbonate-based solvent prepared by mixing together ethylene carbonate (EC):ethylmethyl carbonate (EMC):dimethyl carbonate (DMC)=20:40:40 in a volume ratio was used.

The non-aqueous organic solvent was mixed together with a 1.15 M lithium salt (LiPF₆), and 0.10 wt % of a first additive represented by Chemical Formula 1-1 (pentaerythritol spirobicyclic sulfate, CAS No. 201419-80-9) and 1.00 wt % of a second additive represented by Chemical Formula 3-1 (a weight average molecular weight: 100,000 g/mol), preparing an electrolyte of Example 1.

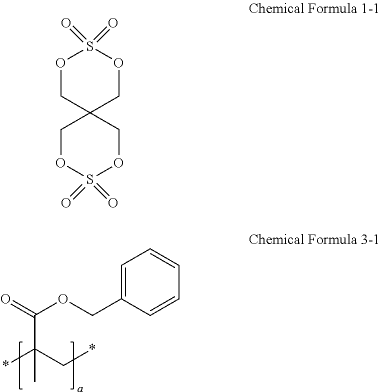

In Chemical Formula 3-1, a is an integer of 1 to 100.

(“wt %” in the composition of the electrolyte is based on a total content of the electrolyte (lithium salt+non-aqueous organic solvent+additive).)

### (2) Manufacture of Rechargeable Lithium Battery Cells

LiNi_{0.91}Co_{0.04}Al_{0.05}O₂ as a positive electrode active material, polyvinylidene fluoride as a binder, and ketjen black as a conductive material were mixed together respectively in a weight ratio of 98.5:0.75:0.75, and then, dispersed in N-methyl pyrrolidone to prepare positive electrode active material slurry.

The positive electrode active material slurry was coated on a 14 μm-thick A1 foil, dried at 110° C., and pressed to manufacture a positive electrode.

A mixture of artificial graphite and silicon particles in a weight ratio of 93.5:6.5 was prepared as a negative electrode active material, and the negative electrode active material, a styrene-butadiene rubber binder, and carboxylmethyl cellulose in a weight ratio of 97:1:2 were dispersed in distilled water to prepare negative electrode active material slurry.

The negative electrode active material slurry was coated on a 10 μm-thick Cu foil, dried at 100° C., and pressed to manufacture a negative electrode.

The manufactured positive and negative electrodes were assembled with a 25 μm-thick polyethylene separator to manufacture an electrode assembly, and after housing the electrode assembly into a pouch with a width of 5.9 cm, a length of 7.8 cm, and a thickness of 5.4 mm as a battery case, and the electrolyte of Example 1 was injected thereinto, manufacturing a rechargeable lithium battery cell.

### EXAMPLES 2 TO 10 AND COMPARATIVE EXAMPLES 1 TO 11

Each additive, electrolyte, and rechargeable lithium battery cell of Examples 2 to 10 and Comparative Examples 1 to 11 was manufactured in substantially the same manner as in Example 1 except that the weight ratio of two types of additive and the total content of the additives were changed as shown in Table 1.

For reference, Comparative Example 1 used no additives at all, Comparative Examples 2 to 6 used the first additive alone, and Comparative Examples 7 to 11 used the second additive alone.

TABLE 1
unit: wt %

| | First additive | Second additive | |
|---|---|---|---|
| | (Chemical | (Chemical | Total |
| | Formula 1) | Formula 3) | additives |
| Comp. Ex. 1 | — | — | — |
| Comp. Ex. 2 | 0.10 | — | 0.10 |
| Comp. Ex. 3 | 0.25 | — | 0.25 |
| Comp. Ex. 4 | 0.50 | — | 0.50 |
| Comp. Ex. 5 | 1.00 | — | 1.00 |
| Comp. Ex. 6 | 2.00 | — | 2.00 |
| Comp. Ex. 7 | — | 1.00 | 1.00 |
| Comp. Ex. 8 | — | 3.00 | 3.00 |
| Comp. Ex. 9 | — | 5.00 | 5.00 |
| Comp. Ex. 10 | — | 10.00 | 10.00 |
| Comp. Ex. 11 | — | 15.00 | 15.00 |
| Example 1 | 0.10 | 1.00 | 1.10 |
| Example 2 | 0.10 | 3.00 | 3.10 |
| Example 3 | 0.10 | 5.00 | 5.10 |
| Example 4 | 0.10 | 10.00 | 10.10 |
| Example 5 | 0.10 | 15.00 | 15.10 |
| Example 6 | 0.25 | 1.00 | 1.25 |
| Example 7 | 0.25 | 3.00 | 3.25 |
| Example 8 | 0.25 | 5.00 | 5.25 |
| Example 9 | 0.25 | 10.00 | 10.25 |
| Example 10 | 0.25 | 15.00 | 15.25 |
| Example 11 | 0.50 | 1.00 | 1.50 |
| Example 12 | 0.50 | 3.00 | 3.50 |
| Example 13 | 0.50 | 5.00 | 5.50 |
| Example 14 | 0.50 | 10.00 | 10.50 |
| Example 15 | 0.50 | 15.00 | 15.50 |
| Example 16 | 1.00 | 1.00 | 2.00 |
| Example 17 | 1.00 | 3.00 | 4.00 |
| Example 18 | 1.00 | 5.00 | 6.00 |
| Example 19 | 1.00 | 10.00 | 11.00 |
| Example 20 | 1.00 | 15.00 | 16.00 |
| Example 21 | 2.00 | 1.00 | 3.00 |
| Example 22 | 2.00 | 3.00 | 5.00 |
| Example 23 | 2.00 | 5.00 | 7.00 |
| Example 24 | 2.00 | 10.00 | 12.00 |
| Example 25 | 2.00 | 15.00 | 17.00 |

### EXAMPLE 26

An electrolyte and a rechargeable lithium battery cell were manufactured in substantially the same manner as in Example 1 except that the electrolyte of Example 1 was prepared by using an additive represented by Chemical Formula 2-1 (1,3-Propane sulfone, CAS NO (1120-71-4)) instead of the additive represented by Chemical Formula 1-1.

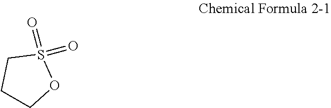

### Evaluation Example: Heat Exposure Evaluation

### (1) Evaluation of 140° C. or 142° C. Heat Exposure Situation

Each rechargeable lithium battery cell according to the examples and the comparative examples was evaluated with respect to heat exposure situation.

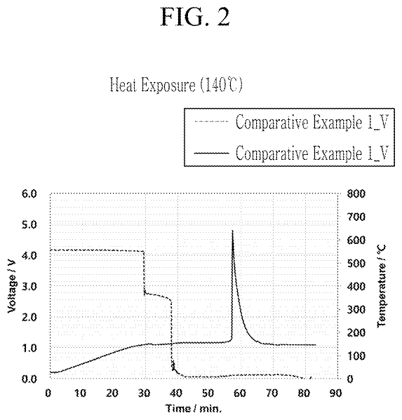

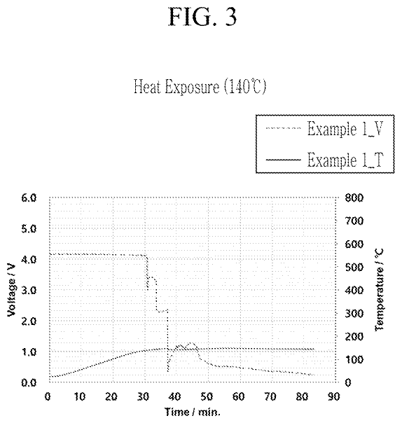

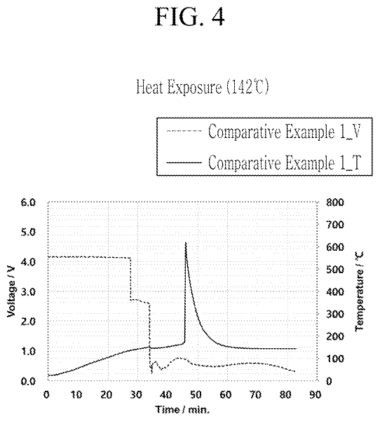

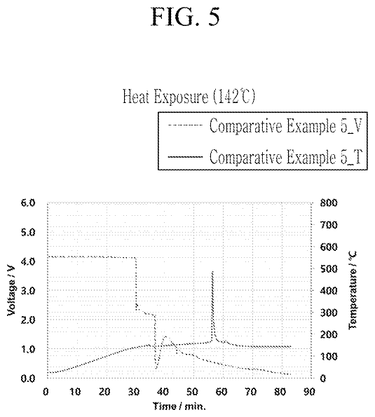

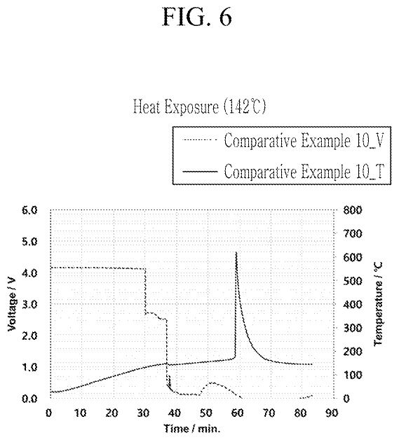

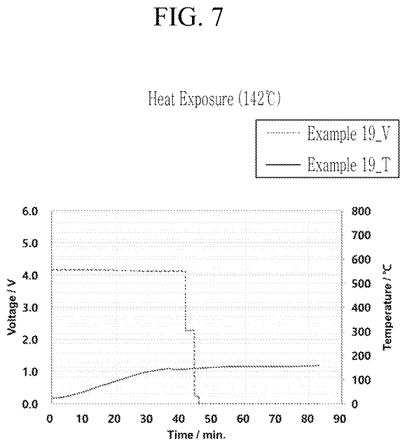

Specifically, each rechargeable lithium battery cell was heated from room temperature to 140° C. or 142° C. at 5° C./min. and maintained at the reached temperature for 1 hour for exposure to heat. Then, the cells were evaluated with respect to a voltage (V) according to time (min.), and the results are shown in FIGS. 2-7.

Referring to FIGS. 2-7, even though one type of additive from the 2 types of additives was used, the heat up to 140° C. was blocked, but if the two types of additives were used in combination, the heat was blocked even up to greater than 140° C. (e.g., 142° C.).

### (2) Heat Exposure Test Pass Temperature and High-Temperature Initial Resistance

Each rechargeable lithium battery cell of the examples and the comparative examples was evaluated with respect to a heat exposure test pass temperature and initial resistance at room temperature, and the results are shown in Table 2.

Heat exposure test pass temperature: Each rechargeable lithium battery cell was heated from room temperature at 5° C./min. to reach 139° C. or higher and allowed to stand at the reached temperature for 1 hour, wherein the reached temperature was evaluated as the “heat exposure test pass temperature.”

Room temperature initial resistance: Each rechargeable lithium battery cell was evaluated under conditions of 25° C. and SOC 100% in a DC resistance method.

TABLE 2

| | Heat exposure test pass | Initial resistance |
|---|---|---|
| | temperature (° C.) | @ SOC100% (mΩ) |
| Comp. Ex. 1 | 138 | 25 |
| Comp. Ex. 2 | 139 | 25 |
| Comp. Ex. 3 | 140 | 25 |
| Comp. Ex. 4 | 140 | 26 |
| Comp. Ex. 5 | 140 | 26 |
| Comp. Ex. 6 | 140 | 28 |
| Comp. Ex. 7 | 139 | 26 |
| Comp. Ex. 8 | 140 | 27 |
| Comp. Ex. 9 | 140 | 28 |
| Comp. Ex. 10 | 140 | 33 |
| Comp. Ex. 11 | 140 | 86 |
| Ex. 1 | 142 | 27 |
| Ex. 2 | 142 | 28 |
| Ex. 3 | 143 | 29 |
| Ex. 4 | 143 | 34 |
| Ex. 5 | 144 | 88 |
| Ex. 6 | 142 | 28 |
| Ex. 7 | 143 | 29 |
| Ex. 8 | 143 | 30 |
| Ex. 9 | 144 | 35 |
| Ex. 10 | 144 | 90 |
| Ex. 11 | 143 | 28 |
| Ex. 12 | 143 | 29 |
| Ex. 13 | 144 | 31 |
| Ex. 14 | 144 | 34 |
| Ex. 15 | 144 | 93 |
| Ex. 16 | 143 | 29 |
| Ex. 17 | 143 | 31 |
| Ex. 18 | 144 | 33 |
| Ex. 19 | 144 | 36 |
| Ex. 20 | 144 | 120 |
| Ex. 21 | 143 | 42 |
| Ex. 22 | 144 | 45 |
| Ex. 23 | 144 | 49 |
| Ex. 24 | 144 | 56 |
| Ex. 25 | 144 | 174 |
| Ex. 26 | 142 | 42 |

### (3) Summary

Referring to Table 2, even though one type of additive from the 2 types of additives was used, the heat up to 140° C. was blocked, but the 2 types of additives were used in combination, the heat was blocked even up to a temperature of greater than 140° C. (e.g., greater than or equal to 142° C.).

If the 2 types of additives were used in combination, comprehensively considering the initial resistance at a high temperature, a content of each additive and a total content of the additives should be limited.

In some embodiments, based on a total amount of the electrolyte, the first additive may be used in an amount of about 0.25 wt % to about 1.00 wt %, the second additive may be used in an amount of about 3.00 wt % to about 10.00 wt %, and the total content of the additives may be about 3.25 wt % to about 11.00 wt %.

While the subject matter of this disclosure has been described in connection with what is presently considered to be practical example embodiments, it is to be understood that the present disclosure is not limited to the disclosed embodiments, but, on the contrary, is intended to cover various modifications and equivalent arrangements included within the spirit and scope of the appended claims, and equivalents thereof.

### DESCRIPTION OF SYMBOLS

100: rechargeable lithium battery
10: positive electrode
20: negative electrode
30: separator
110: electrode assembly
120: case
130: electrode tab

What is claimed is:

1. An electrolyte for a rechargeable lithium battery, comprising:
a non-aqueous organic solvent;
a lithium salt;
a first additive comprising at least one selected from an additive represented by Chemical Formula 1 and an additive represented by Chemical Formula 2; and
a second additive represented by Chemical Formula 3: 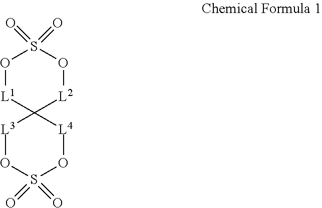
wherein, in Chemical Formula 1,
L¹ to L⁴ are each independently a single bond or a substituted or unsubstituted C1 to C10 alkylene group; 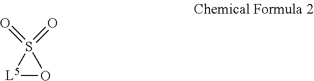
wherein, in Chemical Formula 2,
L⁵ is a single bond or a substituted or unsubstituted C1 to C10 alkylene group; 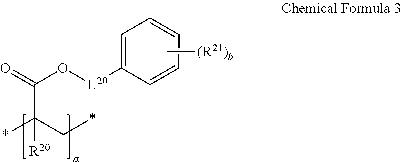
wherein, in Chemical Formula 3,
R²⁰ and R²¹ are each independently a hydrogen atom, a halogen atom, a substituted or unsubstituted C1 to C10 alkyl group, a vinyl group, a substituted or unsubstituted C6 to C10 aryl group, or a substituted or unsubstituted C2 to C10 heteroaryl group including a N, O, or P heteroatom;
L²⁰ is a single bond or a substituted or unsubstituted C1 to C10 alkylene group;
a is an integer of 1 to 100; and
b is an integer of 0 to 5.

2. The electrolyte as claimed in claim 1, wherein
in Chemical Formula 1,
L¹ to L⁴ are all substituted or unsubstituted C1 to C10 alkylene groups.

3. The electrolyte as claimed in claim 1, wherein
in Chemical Formula 2,
L⁵ is a substituted or unsubstituted C1 to C10 alkylene group.

4. The electrolyte as claimed in claim 1, wherein
the first additive comprises at least one of an additive represented by the following formula 1-1 and an additive represented by the following formula 2-1:

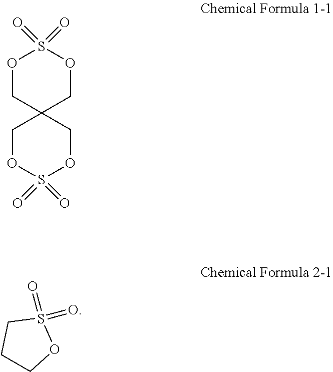 [SMILES: O=S1(=O)OCC2(CO1)COS(=O)(=O)OC2 ; O=S1(=O)CCCO1]

5. The electrolyte as claimed in claim 1, wherein
in Chemical Formula 3,
R²⁰ and R²¹ are each independently a hydrogen atom; or a substituted or unsubstituted C1 to C10 alkyl group.

6. The electrolyte as claimed in claim 1, wherein
in Chemical Formula 3,
L²⁰ is a substituted or unsubstituted C1 to C10 alkylene group.

7. The electrolyte as claimed in claim 1, wherein Chemical Formula 3 is represented by the following Chemical Formula 3-1: 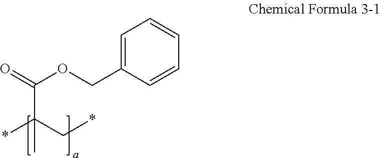
wherein, in Chemical Formula 3-1,
a is an integer of 1 to 100.

8. The electrolyte as claimed in claim 1, wherein
a weight average molecular weight of the second additive is about 5,000 to about 1,000,000 g/mol.

9. The electrolyte as claimed in claim 1, wherein
a weight ratio of the first additive to the second additive is about 1:2 to about 1:20.

10. The electrolyte as claimed in claim 1, wherein
the first additive is included in an amount of about 0.10 to about 2.00 wt % based on a total amount of the electrolyte.

11. The electrolyte as claimed in claim 1, wherein
the second additive is included in an amount of about 1.00 to about 15.00 wt % based on a total amount of the electrolyte.

12. A rechargeable lithium battery, comprising:
a positive electrode comprising a positive electrode active material;
a negative electrode comprising a negative electrode active material; and
the electrolyte as claimed in claim 1.

13. The rechargeable lithium battery as claimed in claim 13, wherein
the positive electrode active material comprises a lithium nickel-based composite oxide represented by Chemical Formula A1:
Liₐ₁Niₓ₁M¹_{y1}M²_{z1}O_{2-b1}X_{b1} Chemical Formula A1
wherein, in Chemical Formula A1,
0.9≤a1≤1.2, 0.7≤x1≤1, 0≤y1≤0.2, 0≤z1≤0.2, 0.9≤x1+y1+z1≤1.1, and 0≤b1≤0.1;
M¹ and M² are each independently one or more elements selected from Al, B, Ba, Ca, Ce, Co, Cr, Cu, Fe, Mg, Mn, Mo, Nb, Si, Sr, Ti, V, W, and Zr; and
X is one or more elements selected from F, P and S.

14. The rechargeable lithium battery as claimed in claim 13, wherein
a separator between the positive electrode and the negative electrode and impregnated with the electrolyte is further included.
